(19) United States
(12) Patent Application Publication
(10) Pub. No.: US 2026/0259115 A1
(43) Pub. Date: Sep. 3, 2026

(54) Continuous bioaerosol sampling device and method

(71) Applicant: THE FIRST AFFILIATED HOSPITAL OF GUANGZHOU MEDICAL UNIVERSITY (GUANGZHOU RESPIRATORY CENTER), Guangzhou (CN)

(72) Inventors: Shaoqiang LI, Guangzhou (CN); Mingdie WANG, Guangzhou (CN); Shiyue LI, Guangzhou (CN); Weilong LI, Guangzhou (CN)

(21) Appl. No.: 19/380,634

(22) Filed: Nov. 5, 2025

Related U.S. Application Data
(63) Continuation of application No. PCT/CN2025/077627, filed on Feb. 17, 2025.

(30) Foreign Application Priority Data
Jan. 17, 2025 (CN) 202510077853.3

(51) Int. Cl. G01N 1/24; G01N 1/22; G01N 33/00; G01N 33/497

(52) U.S. Cl. CPC G01N 1/24; G01N 1/2273; G01N 33/0011; G01N 33/0062; G01N 33/0073; G01N 33/497; G01N 2001/245

(57) ABSTRACT

A continuous bioaerosol sampling device includes a sampling component, a liquid replenishing component, and a control component, where the sampling component includes a sampling cup, a sampling head, and a fan configured to drive the ambient air from the air-outlet passage of the sampling head to form an air flow spirally flowing along an axis of the sampling cup, allow the particulate matters to mix and react with the sampling solution in the sampling cup to form a sample solution, and further allow the remaining air flow to flow outside through the air-outlet passage of the sampling head; the liquid replenishing component is configured to supply the sampling solution from a liquid storage container to the sampling cup through a pump; and the control component is configured to control the pump and the fan according to signals sent from a liquid shortage sensor and a liquid level sensor.

### CROSS REFERENCE TO RELATED APPLICATIONS

The present application is a continuation of International Application No. PCT/CN2025/077627, filed on Feb. 17, 2025, which claims priority from Chinese Application No. 202510077853.3 filed on Jan. 17, 2025, all of which are hereby incorporated herein by reference.

### TECHNICAL FIELD

The present disclosure relates to the field of medical devices, and in particular, to a continuous bioaerosol sampling device and method.

### BACKGROUND

Collecting and detecting bioaerosols in the air is an important means for monitoring the spread of pathogenic microorganisms and bioaerosols in the air. To avoid the omission of aerosol collection caused by changes in bioaerosols in hospitals, bio-pharmaceutical workshops, laboratories, and other places, it is necessary to conduct long-term continuous sampling of aerosols.

However, the current bioaerosol collection device cannot achieve long-term automatic sampling. The main reasons are in that a filter-based sampling device experiences a significant reduction in collection efficiency once the aerosol collected by a filter membrane becomes saturated, and the filter membrane must be replaced and treated through elution before detection on collected samples; and a conventional wet-type sampling device faces difficulties in long-term automatic sampling due to the uncertainty of liquid evaporation, and cannot provide real-time state monitoring and control of the device.

The problems of missing detection in existing bioaerosol collection, the inability of collection devices to perform long-term continuous sampling, the need for intermittent liquid replenishing for further sampling, the lack of remote monitoring and real-time control of sampling devices, and the inability of automatic replenishment of sampling solutions lead to low collection efficiency of bioaerosols, which needs to be solved urgently.

### SUMMARY

The present disclosure therefore provides an improved continuous bioaerosol sampling device and method, which is free from the above-mentioned problems.

One aspect of the invention provides a continuous bioaerosol sampling device, including:
a sampling component configured for collecting a sample solution, including:
   a sampling cup configured for receiving a sampling solution which is mixed with particulate matters in ambient air to form the sample solution;
   a sampling head connected to the sampling cup, which is formed with a circumferential air-inlet passage corresponding to a peripheral area of the sampling cup and a central air-outlet passage corresponding to a center area of the sampling cup; and
   a fan connected to the sampling head, with an air suction end of the fan in air communication with the circumferential air-inlet passage and an air exhaust end of the fan in air communication with the central air-outlet passage, wherein the fan is configured to drive the ambient air entering the sampling head to pass through the central air-outlet passage and form an air flow spirally flowing along an axis of the sampling cup, allow the particulate matters in the air flow to mix and react with the sampling solution in the sampling cup to form the sample solution, and further allow the remaining air flow entering the sampling cup to flow to the central air-outlet passage of the sampling head from the center area of the sampling cup, which is finally discharged from the air exhaust end of the fan;
a liquid replenishing component, including:
   a liquid storage container for storing the sampling solution; and
   a pump for supplying or replenishing the sampling solution from the liquid storage container to the sampling cup; and
a control component, including:
   a liquid shortage sensor arranged between the liquid storage container and the pump, which is configured for detecting whether the sampling solution in the liquid storage container is supplied to the sampling cup; and
   a liquid level sensor for detecting a liquid level of the sampling solution in the sampling cup;
   wherein the control component is configured to control the pump to supply or replenish sampling solution form the liquid storage container to the sampling cup, in a response to a signal detected by the liquid shortage sensor indicating that the sampling solution in the liquid storage container is supplied to the sampling cup and a signal detected by the liquid level sensor indicating that the liquid level in the sampling cup is less than a predefined threshold value, and control the pump and the fan to inactivate, in a response to a signal detected by the liquid shortage sensor indicating that the sampling solution in the liquid storage container is not supplied to the sampling cup or a signal detected by the liquid level sensor indicating that the liquid level in the sampling cup reaches the predefined threshold value.

Another aspect of the invention provides continuous bioaerosol sampling method, including steps of:
activating, by the control component, the pump to run;
determining, by the liquid shortage sensor, whether the sampling solution is supplied to the sampling cup;
continues running, by the control component, the pump for a period of time to supply the sampling solution to the sampling cup, in response to the signal detected by the liquid level sensor indicating that the liquid level in the sampling cup is less than the predefined threshold value;
inactivating, by the control component, the pump to stop running, in response to the signal detected by the liquid level sensor indicating that the liquid level in the sampling cup reaches the predefined threshold value;
activating the fun, by the control component, to drive the ambient air entering the sampling head to mix and react with the sampling solution in the sampling cup to form the sample solution, and further allow the remaining air flow entering the sampling cup to be discharged from the air exhaust end of the fan;
reactivating, by the control component, the pump to replenish the sampling solution to the sampling cup during sampling, until the liquid level in the sampling cup reaches the predefined threshold value, in response to the signal detected by the liquid level sensor indicating that the liquid level in the sampling cup is less than the predefined threshold value; and
inactivating the fun and the pump by the control component, in response to the signal detected by the liquid shortage sensor indicating that the sampling solution in the liquid storage container is not supplied to the sampling cup.

### BRIEF DESCRIPTION OF THE DRAWINGS

FIG. 1 is a schematic structural diagram of a sampling device according to the present disclosure;

FIG. 2 is a view schematically showing an air transport path in the sampling device;

FIG. 3 is a view schematically showing the liquid transport path in the sampling device;

FIG. 4 is a top view of a sampling head of the sampling device;

FIG. 5 is a side view of the sampling head;

FIG. 6 is a view schematically showing a sampling cup of the sampling device; and

FIG. 7 shows a schematic diagram of a circuit control in the sampling device.

### DETAILED DESCRIPTION OF THE EMBODIMENTS

The accompanying drawings of the present disclosure are only intended for illustrative purposes and should not be construed as limiting the present disclosure. In order to better illustrate the following embodiments, some components in the accompanying drawings may be omitted, enlarged or reduced, and do not represent actual product dimensions. It will be understood by those having ordinary skill in the art that certain well-known structures and their descriptions in the accompanying drawings may be omitted.

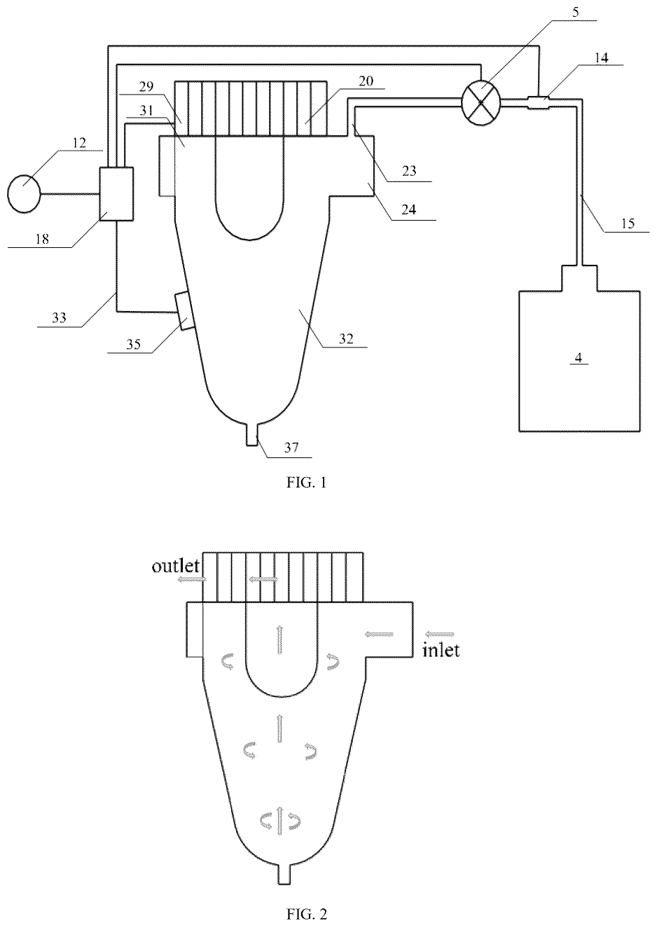

FIG. 1 depicts a continuous bioaerosol sampling device, including a sampling component, a liquid replenishing component, and a control component. The sampling component is configured for collecting a sample solution, which particularly includes a sampling cup 32 for receiving a sampling solution, a sampling head 31 connected to the sampling cup 32, and a fan 20 connected to the sampling head 31. The sampling head 31 is formed with a circumferential air-inlet passage 311 in air communication with an air suction end of the fan 20 and a central air-outlet passage 312 in air communication with an air exhaust end of the fan 20. Under the action of the fan 20, along the circumferential air-inlet passage 311 the ambient air entering the sampling head 31 becomes into an accelerated flow which is spirally and axially flowed to the bottom of the sampling cup 32 along the inner wall of the sampling cup 32, namely a downward cyclone rotated around the axial of the sampling cup 32 is formed in the sampling cup 32. During rotation, the particulate matters in the air flow are thrown to the inner wall of the sampling cup 32 due to the centrifugal force, which mix and react with the sampling solution to form the sample solution. Under the action of the fan 20, the rest of the air flow entering the sampling cup 32 is flowed to the central air-outlet passage 312 from the center of the sampling cup 32, namely an upward cyclone is formed, which is finally discharged from the air exhaust end of the fan 20. The liquid replenishing component includes a liquid storage container 4 for storing the sampling solution and a peristaltic pump 5 for supplying or replenishing the sampling solution from the liquid storage container 4 to the sampling cup 32. The control component includes a liquid shortage sensor 14 for detecting whether the sampling solution in the liquid storage container 4 is supplied to the sampling cup 32 and a liquid level sensor for detecting the liquid level of the sampling solution in the sampling cup 32. The liquid shortage sensor 14 is preferably arranged between the liquid storage container 4 and the peristaltic pump 5. The liquid level sensor is arranged on a side wall of the sampling cup 32. The control component is configured to control the operation state of the peristaltic pump 5 and the fan 20 according to signals sent from the liquid shortage sensor 14 and the liquid level sensor.

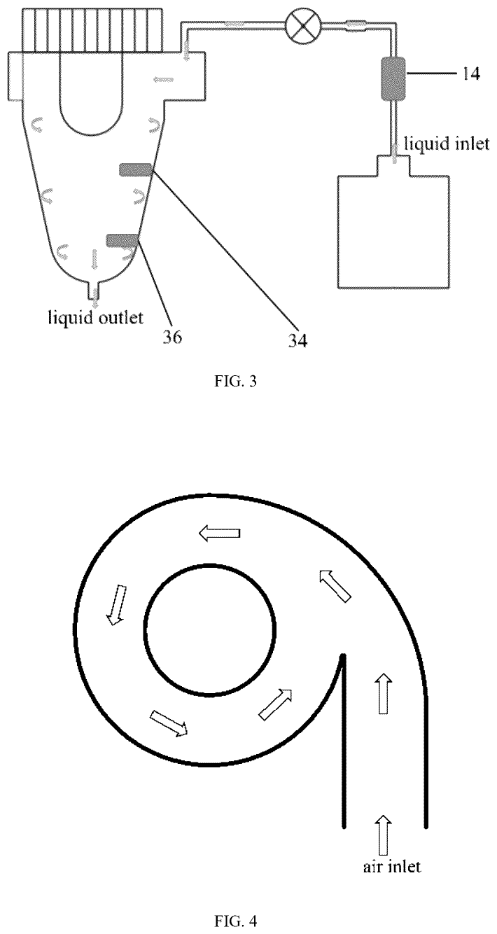

In this embodiment, both air transport path and liquid transport path are involved in the sampling cup 32. FIG. 2 is a view schematically showing the air transport path and FIG. 3 is a view schematically showing the liquid transport path. In FIG. 2, the flow direction of the air flow is indicated by arrows. For sampling, the fan 20 is activated to accelerate the air flow entering the sampling head 31 and allow the accelerated air flow to flow toward the sampling cup 32 along the circumferential air-inlet passage 311 to form an outer swirling flow which is flowed downwardly in the axial direction of the sampling cup 32. Under the action of the fan 20, the airflow entering the sampling cup 32 is flowed upwardly in the axial direction of the sampling cup 32 to form an inner swirling flow which is finally discharged from the air outlet 29 in the air exhaust end of the fan 20 through the central air-outlet passage 312 of the sampling head 31.

In FIG. 3, the flow direction of the liquid flow is indicated by arrows. For sampling, the peristaltic pump 5 is activated to transport the sampling solution from the liquid storage container 4 to the sampling cup 32. The liquid level sensor detects the liquid level of the sampling solution in the sampling cup 32 in real time, where when the liquid level sensor detects that the liquid level of the sampling solution in the sampling cup 32 has reached the predefined threshold value, the peristaltic pump 5 is inactivated by the control component. During sampling, when the liquid level sensor detects that the liquid level of the sampling solution in the sampling cup 32 is less than the predefined threshold value, the peristaltic pump 5 is activated by the control component to replenish the sampling solution to the sampling cup 32 until the liquid level reaches the predefined threshold value, thereby achieving continuous sampling. In addition, during sampling the liquid shortage sensor 14 detects whether the sampling solution is supplied from the liquid storage container 4 to the sampling cup 32, where when the liquid shortage sensor 14 detects that the sampling solution is not supplied, the fan 20 and the peristaltic pump 5 are inactivated by the control component to stop sampling. This ensures normal operation of the sampling device. Sampling restarts with the restoring of sampling solution supplying, such as by adding sampling solution into the liquid storage container 4 or replacing a new liquid storage container 4, if the abnormal sampling solution supplying is caused by inadequate sampling solution of the liquid storage container 4.

In this embodiment, the fan 20 provides an air power source for the sampling of the sampling device. The blades of the fan 20 rotating at a high speed accelerates the air flow to spirally flow downward along the inner wall of the device towards the sampling cup 32. Under the action of the centrifugal force generated by the rotating flow, the particulate matters in the air flow are thrown to the inner wall of the sampling cup 32, which finally mix and react with the sampling solution to form the sample solution, thus achieving sample collection. Under the action of the fan 20, the remaining air flow entering the sampling cup 32 is flowed upwardly at the centre of the the sampling cup 32 and finally discharged from the air outlet 29 of the air exhaust end of the fan 20 through the central air-outlet passage 312 of the sampling head 31.

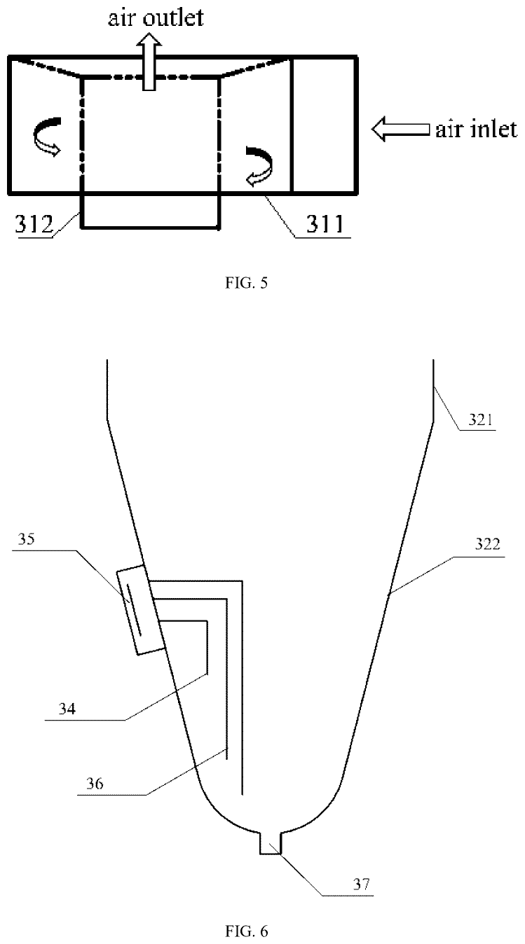

FIG. 4 depicts a top view of the sampling head 31 and FIG. 5 depicts a side view of the sampling head 31. In combination with FIG. 4 and FIG. 5, one side wall of the sampling head 31 extends outward to form an air inlet 24 in air communication with the circumferential air-inlet passage 311. The sampling head 21 has an inner recessed portion at the central portion which forms the central air-outlet passage 312. The circumferential air-inlet passage 311 is defined by the inner recessed portion and the side wall of the sampling head 31. As shown, the ambient air tangentially flows into the circumferential air-inlet passage 311 through the air inlet 24, which allows the airflow spirally flow to the sampling cup 32. In such configuration, the collection efficiency of the particulate matters in the air flow is improved. The sampling head 31 in the preset embodiment is substantially in cylindrical shape.

FIG. 6 schematically illustrates the structure of the sampling cup 32. The sampling cup 32 in the present embodiment includes a cylindrical transition segment 321 and a sample collecting segment 322. The sample collecting segment 322 has a diameter gradually decreasing from the transition segment 321 to the end. The end of the sample collecting segment 322 has a liquid outlet 37. The cylindrical transition segment 321 guides the accelerated air flow from the circumferential air-inlet passage 311 of the sampling head 31 into the sampling cup 32 to form downward cyclone. In the sample collecting segment 322, the particulate matters are divided from the accelerated spiral air flow under the action of the centrifugal force. The decreased diameter of the sample collecting segment 322 further accelerates the air flow and allows the particulate matters to quickly mix with the sampling solution in the sampling cup 32, which improves the sample collecting efficiency. Through the liquid outlet 37, the sample solution collected in the sampling cup 32 can be obtained at any time without disassembly of the device.

In order to ensure continuous operation of the device, the liquid level sensor includes a low liquid level sensor 36 and a high liquid level sensor 34, which detects the liquid level in the sampling cup 32 in real time, thereby keeping the liquid volume within the specified range and ensuring normal sampling. The low liquid level sensor 36 allows the liquid volume to keep within the specified range by limiting the liquid volume to a minimum liquid volume, and the high liquid level sensor 34 limits a maximum liquid volume of the sampling solution inside the sampling cup 32 to avoid excessive liquid volume which influences normal sampling.

Referring back to FIG. 1, the sampling head 31 is provided with a liquid replenishing port 23 close to the air inlet 24. The sampling solution is supplied to the sampling cup 32 through the liquid replenishing port 23. With such configuration, the sampling solution entering the sampling cup 32 immediately mix with the particulate matters attached to the inner wall of the sampling cup 32, which can shorten the sampling time and further improving sample collecting efficiency.

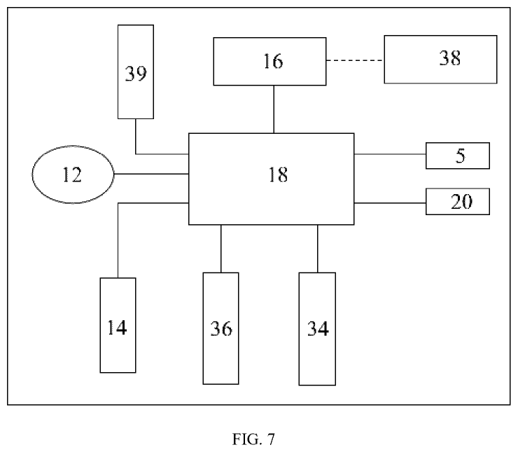

FIG. 7 illustrates a schematic diagram of a circuit control in the sampling device. As shown, the control component further includes a control circuit board 18 and a fault indicator light 39. The fault indicator light 39 is configured to indicate an operation fault when no sampling solution is transported to the sampling cup 32 and the sampling solution in the sampling cup 32 reaches the predefined maximum threshold value. The control circuit board 18 is communicatively connected to the peristaltic pump 5, the fan 20, the fault indicator light 39, the liquid shortage sensor 14, and the liquid level sensor.

In combination with FIG. 1, via interface 35 of the sampling cup 32, especially an USB interface, the low liquid level sensor 36 and the high liquid level sensor 34 are communicatively connected to the control circuit board 18 through a single line 33, so that the control circuit board 18 controls operation of the peristaltic pump 5 and the fan 20 according to the signal detected by the low liquid level sensor 36 and the high liquid level sensor 34, which keeps the sampling solution of the sampling cup 32 in a certain range to ensure normal sampling.

In the present embodiment, when the sampling solution in the sampling cup 32 is not enough, namely the liquid level has not reached the minimum threshold value, the low liquid level sensor 36 detects a liquid level signal indicating that the sampling solution is less than the minimum threshold value, and sends such signal to the control circuit board 18 so as to control the pump 5 to activate and transport sampling solution to the sampling cup 32 from the liquid storage container 4. When the low liquid level sensor 36 detects a liquid level signal indicating that the sampling solution reaches the minimum threshold value, and sends such signal to the control circuit board 18 so as to control the pump 5 to inactivate and stop transporting sampling solution to the sampling cup 32 from the liquid storage container 4, thereby keeping the sampling solution in the sampling cup in a certain range for normal sampling. In a case that the low liquid level sensor 36 is abnormal, which cannot send liquid level signal to the control circuit board 18 to stop transporting sampling solution to the sampling cup 32, the sampling solution in the sampling cup 32 will increase continuously until the high liquid level sensor 34 detects a liquid signal indicating that the liquid level in the sampling cup 32 has reached the maximum threshold, where the control circuit board 18 controls the pump 5 to stop transporting sampling solution to the sampling cup 32, thereby avoiding excess liquid level. In addition, when the liquid level in the sampling cup 32 has reached the maximum threshold, the fault indicator light 39 lights up to indicate a fault signal.

When the liquid shortage sensor 14 detects a signal indicating that the sampling solution is not supplied from the liquid storage container 4 to the sampling cup 32. The control circuit board 18 controls the pump 5 and the fan 20 to inactive when receiving the liquid shortage signal from the liquid shortage sensor 14. In addition, the fault indicator light 39 lights up to indicate a fault signal which reminds the users of the abnormal sampling solution supplying.

In a preferable embodiment, the sampling solution is transported to the sampling cup 32 through a conduit 15, especially a hose, namely the liquid storage container 4, the pump 5, and the liquid replenishing port 23 are connected each other through the conduit 15. More preferably, an additional liquid shortage sensor 14 is further provided in the conduit 15 close to the liquid storage container 4 and the liquid replenishing port 23.

In this embodiment, the liquid shortage sensor 14 detects in real time whether the sampling solution is flowed in the conduit 15. When the liquid shortage sensor 14 detects that the sampling solution is present in the conduit 15, the sampling device operates normally; and when the liquid shortage sensor 14 detects that no sampling solution is present in the conduit 15, the liquid shortage sensor 14 sends an alarm signal to the control circuit board 18, the control circuit board 18 stops operation of the sampling device, and the fault indicator light 39 lights up to indicate abnormal sampling solution supplying.

In this embodiment, in order to quickly determine the fault location, in addition to the liquid shortage sensor 14 close to the peristaltic pump 5, an additional liquid shortage sensor 14 is also arranged at a connection port between the conduit 15 and the liquid storage container 4, and a connection port between the conduit 15 and the liquid replenishing port 23 of the sampling cup 32. These sensors can determine whether there is liquid flow in different sections of the conduit 15, thereby quickly identifying the specific fault location in a case that the fault indicator light 39 lights up. For example, the sensor close to the liquid storage container 4 can determine whether the abnormal sampling solution supplying is caused by inadequate sampling solution in the liquid storage container 4 by detecting whether there is liquid shortage signal. The sensor close to the liquid replenishing port 23 can determine whether the abnormal sampling solution supplying is caused by the fault of the liquid shortage sensor 14 by detecting whether there is liquid shortage signal.

In this embodiment, the capacity of the liquid storage container 4 may be designed according to the implementation situation. The evaporation rate of the sampling solution usually increases with higher temperatures and lower humidity. Before the capacity of the liquid storage container 4 is customized, a table showing the evaporation rate of the sampling solution under different temperature and humidity conditions is obtained. The required amount of pre-stored sampling solution in the liquid storage container 4 can be calculated according to the local temperature and humidity and the actual sampling time. For example, if the evaporation rate of the sampling solution is 0.5 ml/min at a temperature of 25° C. and a humidity of 30 RH, the expected sampling time is 3 days, at least 2.16 liters of sampling solution should be pre-stored in the liquid storage container 4 (3*24*60*0.5/1000=2.16).

According to the sampling device of the present embodiment, when the device is plugged into a power supply 12 and a control switch is turned on, the control circuit board 18 controls the peristaltic pump 5 to run according to a sampling command. If the liquid shortage sensor 14 detects a liquid signal in the conduit 15, the pump 5 runs continuously until the low liquid level sensor 36 in the sampling cup 32 detects the liquid signal indicating that the liquid level in the sampling cup 32 has reached the predefined threshold. The control circuit board 18 controls the fan 20 to run, the ambient air is driven to enter the circumferential air-inlet passage 311 through the air inlet 24. Under the action of the fan 20, the air flow is accelerated and twirled downwardly along the axial of the sampling cup 32, allowing the particulate matters in the air flow to mix and react with the sampling solution to form the sample solution. The air flow entering the sampling cup 32 then is flowed to the central air-outlet passage 312 and finally discharged from the air exhaust end of the fan 20. During sampling, when the low liquid level sensor 36 detects the liquid level signal indicating that the liquid level is less than the predefined minimum threshold, the peristaltic pump 5 is restarted to replenish the sampling solution; and when the low liquid level sensor 36 detects the liquid signal indicating that the liquid level has reached the predefined minimum threshold, the peristaltic pump 5 is stopped from replenishing the sampling solution. If the low liquid level sensor 36 has a fault and liquid replenishing continues until the high liquid level sensor 34 detects the liquid signal, the control circuit board 18 inactivates the fan 20 and the peristaltic pump 5 to stop sampling, and the fault indicator light 39 lights up. During sampling, when the liquid shortage sensor 14 detects the signal indicating that no sampling solution is supplied in the conduit 15, the control circuit board 18 inactivates the fan 20 and the peristaltic pump 5 to stop sampling, and the fault indicator light 39 lights up.

A wireless transmission module 16 communicatively connected to the control circuit board 18 and a remote monitoring system 38 communicatively connected to the wireless transmission module 16 are further included. The wireless transmission module 16 is configured to transmit a wireless signal to the remote monitoring system 38, enabling real-time transmission of operating state data of the sampling device to the remote monitoring system 38.

In this embodiment, the wireless transmission module 16 may be in a wireless Wi-Fi, 4G, 5G communication mode. During sampling operation, all sensors may continuously transmit signals to the control circuit board 18, and the control circuit board 18 may simultaneously transmit signals to the remote monitoring system 38 through the wireless transmission module 16. The remote monitoring system 38 may monitor the operating state of the sampling device, including sampling start time, sampling operation time, sampling stop time, and fault information, and also store the operating state data of the sampling device on a server.

A continuous bioaerosol sampling method conducted by the sampling device in any case mentioned above is further provided according to an embodiment of the present disclosure.

In the method, the pump 5 is activated to run for a period of time by the control component, the liquid shortage sensor 14 detects whether the sampling solution is transported to the sampling cup 32, if yes, the pump 5 continues to run until the liquid level sense detects that the liquid level in the sampling cup 32 has reached the predefined threshold value, then the pump 5 is inactivated by the control component. The fan 20 is activated to run by the control component, which drives the ambient air to enter the circumferential air-inlet passage 311. Under the action of the fan 20, the air flow is accelerated and twirled downwardly along the axial of the sampling cup 32, allowing the particulate matters in the air flow to mix and react with the sampling solution to form the sample solution. The air flow entering the sampling cup 32 then is flowed to the central air-outlet passage 312 and finally discharged from the air exhaust end of the fan 20. During continuous sampling, when the liquid level sensor detects the liquid level in the sampling cup 32 is less than the predefined minimum threshold, the pump 5 is reactivated by the control component to replenish sampling solution until the liquid level in the sampling cup 32 reaches the predefined minimum threshold. When the liquid shortage sensor 14 detects that the sampling solution is not supplied to the sampling cup 32, the pump 5 and the fan 20 are inactivated by the control component.

According to a preferable embodiment, the liquid level sensor includes a low liquid level sensor 36 and a high liquid level sensor 34. During sampling, when the low liquid level sensor 36 detects the liquid level signal indicating that the liquid level is less than the predefined minimum threshold, the peristaltic pump 5 is restarted by the control component to replenish the sampling solution; and when the low liquid level sensor 36 detects the liquid signal indicating that the liquid level has reached the predefined minimum threshold, the peristaltic pump 5 is stopped from replenishing the sampling solution. When the high liquid level sensor 34 detects the liquid signal indicating that the liquid level has reached the predefined maximum threshold, the fan 20 and the pump 5 are inactivated by the control component to stop sampling, and the fault indicator light 39 lights up and send a fault signal indicating abnormal sampling solution supplying.

Supposing that the minimum threshold of the liquid level is a capacity of P (L), t(s) is time required for the peristaltic pump 5 to supply sampling solution to the sampling cup 32,

$$t = 1000 * \frac{P}{V * {S}_{conduit 15}} , where {S}_{conduit 15} ( {m}^{2} )$$

is the cross-sectional area of the conduit 15, and V (m/s) is a flow rate of the sampling solution.

Obviously, the above-mentioned embodiments of the present disclosure are only examples for clearly explaining the technical schemes of the present disclosure, and are not intended to limit the specific implementations of the present disclosure. Any modifications, equivalent replacements and improvements made within the spirit and principle of the claims of present disclosure should be included within the protection scope of the claims of the present disclosure.

What's claimed is:

1. A continuous bioaerosol sampling device, comprising:
a sampling component configured for collecting a sample solution, comprising:
a sampling cup configured for receiving a sampling solution which is mixed with particulate matters in ambient air to form the sample solution;
a sampling head connected to the sampling cup, which is formed with a circumferential air-inlet passage corresponding to a peripheral area of the sampling cup and a central air-outlet passage corresponding to a center area of the sampling cup; and
a fan connected to the sampling head, with an air suction end of the fan in air communication with the circumferential air-inlet passage and an air exhaust end of the fan in air communication with the central air-outlet passage, wherein the fan is configured to drive the ambient air entering the sampling head to pass through the central air-outlet passage and form an air flow spirally flowing along an axis of the sampling cup, allow the particulate matters in the air flow to mix and react with the sampling solution in the sampling cup to form the sample solution, and further allow the remaining air flow entering the sampling cup to flow to the central air-outlet passage of the sampling head from the center area of the sampling cup, which is finally discharged from the air exhaust end of the fan;
a liquid replenishing component, comprising:
a liquid storage container for storing the sampling solution; and
a pump for supplying or replenishing the sampling solution from the liquid storage container to the sampling cup; and
a control component, comprising:
a liquid shortage sensor arranged between the liquid storage container and the pump, which is configured for detecting whether the sampling solution in the liquid storage container is supplied to the sampling cup; and
a liquid level sensor for detecting a liquid level of the sampling solution in the sampling cup;
wherein the control component is configured to control the pump to supply or replenish sampling solution form the liquid storage container to the sampling cup, in a response to a signal detected by the liquid shortage sensor indicating that the sampling solution in the liquid storage container is supplied to the sampling cup and a signal detected by the liquid level sensor indicating that the liquid level in the sampling cup is less than a predefined threshold value, and control the pump and the fan to inactivate, in a response to a signal detected by the liquid shortage sensor indicating that the sampling solution in the liquid storage container is not supplied to the sampling cup or a signal detected by the liquid level sensor indicating that the liquid level in the sampling cup reaches the predefined threshold value.

2. The continuous bioaerosol sampling device according to claim 1, wherein the sampling head has an air inlet extending outward at one side wall, which is in air communication with the circumferential air-inlet passage, and an inner recessed portion at a central portion, which defines the central air-outlet passage.

3. The continuous bioaerosol sampling device according to claim 2, wherein the sampling head is provided with a liquid replenishing port close to the air inlet, through which the sampling solution is supplied to the sampling cup.

4. The continuous bioaerosol sampling device according to claim 1, wherein the sampling cup comprises a cylindrical transition segment and a sample collecting segment having a diameter gradually decreasing from the transition segment to an end, and the end of the sample collecting segment has a liquid outlet.

5. The continuous bioaerosol sampling device according to claim 4, wherein an additional liquid shortage sensor is further arranged close to the liquid replenishing port and the liquid storage container.

6. The continuous bioaerosol sampling device according to claim 1, wherein the liquid level sensor includes a low liquid level sensor and a high liquid level sensor, the low liquid level sensor is configured to detect whether the liquid level in the sampling cup is less than a predefined minimum threshold value, if yes, a corresponding signal is sent to the control component to activate or continuously run the pump, if not, a corresponding signal is sent to the control component to inactivate the pump, and the high liquid level sensor is configured to detect whether the liquid level in the sampling cup reaches a predefined maximum threshold value, if yes, a corresponding signal is sent to the control component to inactivate the pump and the fan.

7. The continuous bioaerosol sampling device according to claim 1, wherein the control component further comprises a control circuit board communicatively connected to the pump, the fan, the liquid shortage sensor, and the liquid level sensor, which is configured to control the pump and the fan according to signals received from the liquid shortage sensor and the liquid level sensor.

8. The continuous bioaerosol sampling device according to claim 7, wherein the control component further comprises a fault indicator communicatively connected to control circuit board, which is configured to indicate that the liquid level is not supplied to the sampling cup or the liquid level in the sampling cup reaches a predefined maximum threshold value.

9. The continuous bioaerosol sampling device according to claim 8, wherein the control component further comprises a wireless transmission module communicatively connected to the control circuit board and a remote monitoring system communicatively connected to the wireless transmission module, the wireless transmission module is configured to transmit operating state data of the sampling device to the remote monitoring system.

10. A continuous bioaerosol sampling method conducted by the continuous bioaerosol sampling device according to claim 1, comprising steps of:
activating, by the control component, the pump to run;
determining, by the liquid shortage sensor, whether the sampling solution is supplied to the sampling cup;
continues running, by the control component, the pump for a period of time to supply the sampling solution to the sampling cup, in response to the signal detected by the liquid level sensor indicating that the liquid level in the sampling cup is less than the predefined threshold value;
inactivating, by the control component, the pump to stop running, in response to the signal detected by the liquid level sensor indicating that the liquid level in the sampling cup reaches the predefined threshold value;
activating the fun, by the control component, to drive the ambient air entering the sampling head to mix and react with the sampling solution in the sampling cup to form the sample solution, and further allow the remaining air flow entering the sampling cup to be discharged from the air exhaust end of the fan;
reactivating, by the control component, the pump to replenish the sampling solution to the sampling cup during sampling, until the liquid level in the sampling cup reaches the predefined threshold value, in response to the signal detected by the liquid level sensor indicating that the liquid level in the sampling cup is less than the predefined threshold value; and
inactivating the fun and the pump by the control component, in response to the signal detected by the liquid shortage sensor indicating that the sampling solution in the liquid storage container is not supplied to the sampling cup.

11. The method according to claim 10, wherein in a case that the liquid level sensor includes a low liquid level sensor and a high liquid level sensor, the method comprises:
activating, by the control component, the pump to supply or replenish the sampling solution to the sampling cup until the liquid level in the sampling cup reaches a predefined minimum threshold value, in response to a signal detected by the low liquid level sensor indicating that the liquid level in the sampling cup is less than a predefined minimum threshold value; and
inactivating, by the control component, the pump and the fan, in response to a signal detected by the high liquid level sensor indicating that the liquid level in the sampling cup reaches a predefined maximum threshold value.
